(19) United States
(12) Patent Application Publication
(10) Pub. No.: US 2005/0182302 A1
(43) Pub. Date: Aug. 18, 2005

(54) System, apparatus and method for evaluating health and wellness

(76) Inventors: David Johnson, Round Lake Beach, IL (US); Jeffrey Eicoff, Mundelein, IL (US)

Correspondence Address: PATENTS+TMS, P.C., 2849 W. ARMITAGE AVE., CHICAGO, IL, 60647, US

(21) Appl. No.: 10/779,087

(22) Filed: Feb. 17, 2004

(51) Int. Cl. A61B 5/00; A61B 10/00

(52) U.S. Cl. 600/300; 128/920; 128/903

(57) ABSTRACT

A system, apparatus and method for evaluating the health and wellness of an individual are provided. A portable electronic device, such as, for example, a cellular telephone is provided. Physiological information is received from an individual which may be stored, analyzed and/or displayed. The physiological information may be invasively or non-invasively received from the individual. Information relating to the lifestyle, diet and/or exercise of the individual may also be entered. This information, as with the physiological information, may be stored, analyzed and/or displayed. The system, apparatus and method also may communicate with a cellular system and/or a computer network. An individual may use information received and/or entered to evaluate his/her health or wellness. Other individuals, such as, for example, a healthcare provider, may use the received and/or entered information for similar evaluations.

### BACKGROUND OF THE INVENTION

The present invention relates to a system, apparatus and method for evaluating the health and wellness of an individual. More specifically, the present invention uses received and/or entered information relating to the physiology, lifestyle, diet and/or exercise of an individual to evaluate his/her health or wellness. The present invention may store, analyze and/or display the received and/or entered information. The present invention may also use the received and/or entered information in communication with a cellular system and/or a computer network. The present invention may receive physiological information from an individual in a non-invasive manner.

It is generally known that the health and wellness of an individual is influenced by his/her physiology and environmental factors, such as, for example, lifestyle. Unfortunately, however, given the increasing pace of society, the difficulties in monitoring and evaluating physiology and lifestyle are mounting. Moreover, the quality of a lifestyle may either positively or negatively influence health and wellness. Individuals are commonly unaware of deteriorations in health or wellness which may increase the susceptibility of future health problems and diseases.

Research has demonstrated that providing individuals with information related to physiology and lifestyle can improve his/her health and wellness. Physiological information, such as, for example, the pulse rate and blood pressure of an individual, can guide health and wellness. Similarly, health and wellness is guided by lifestyle information, such as, for example, diet and exercise. An improvement in health and wellness may dramatically decrease the possibility of a potential health condition. Presently, healthcare and insurance infrastructures do not allow individuals to regularly monitor and evaluate physiology or lifestyle. In addition, the individual-oriented systems and apparatuses used to evaluate physiology or lifestyle that are currently available are expensive and cumbersome. These known systems and apparatuses are also intended to be worn by the user which is impractical and may be physically invasive. Finally, these systems and apparatuses do not evaluate both the physiology and lifestyle of an individual.

A need, therefore, exists for a system, apparatus and method by which to portably evaluate health and wellness of an individual. A need further exists for a system, apparatus and method to receive physiological information. A need further exists for a system, method and apparatus to non-invasively receive physiological information from an individual. A need further exists for a system, apparatus and method to evaluate entered information relating to lifestyle. A need further exists for a system, apparatus and method to communicate with a cellular system and/or a computer network.

### SUMMARY OF THE INVENTION

The present invention provides a system, apparatus and method for evaluating the health and wellness of an individual. The present invention further provides a system, apparatus and method that is portable. The present invention further uses received and/or entered information relating to the physiology, lifestyle, diet and/or exercise of an individual to evaluate his/her health or wellness. The present invention may further store, analyze and/or display the received and/or entered information. The present invention further may use the received and/or entered information in communication with a cellular system and/or a computer network. The present invention further may receive physiological information from an individual in a non-invasive manner and/or invasive manner.

In an embodiment of the present invention, a system for evaluating health and wellness of an individual is provided. The system comprises a central processing unit for analyzing a first plurality of data. The system further has a transmission unit connected to the central processing unit for communicating with a cellular system. The system further has a collection port connected to the central processing unit for receiving physiological information from the individual. The system further has a memory unit connected to the central processing unit for storing the first plurality of data wherein the first plurality of data relates to the physiological information. The system also has a screen connected to the central processing unit for displaying a second plurality of data wherein the second plurality of data is derived from the first plurality of data.

In another embodiment, the system further has an input unit connected to the central processing unit for entering the first plurality of data.

In another embodiment of the system, the first plurality of data relates to a diet of the individual.

In another embodiment of the system, the first plurality of data relates to an activity performed by the individual.

In another embodiment of the system, the collection port non-invasively receives the physiological information through epidermal contact of the individual.

In another embodiment of the system, the physiological information is selected from the group consisting of pulse rate, heart rate, blood pressure, glucose level, blood sugar level, skin temperature, body fat and combinations thereof.

In another embodiment, the system further has a tablet connected to the central processing unit for entering the first plurality of data.

In another embodiment of the system, the central processing unit stores mathematical algorithms for analyzing the first plurality of data.

In another embodiment, the system has an output component connected to the central processing unit for communicating with a computer network.

In an embodiment of the present invention, a cellular telephone having an exterior defined by a body that receives and transmits physiological information related to evaluating health and wellness of an individual is provided. The cellular telephone comprises a central processing unit within the body for analyzing a first plurality of data. The cellular telephone further has a biometric port within the body and connected to the central processing unit for receiving the physiological information from the individual. The cellular telephone further has a memory unit within the body and connected to the central processing unit for storing the first plurality of data wherein the first plurality of data relates to the physiological information. The cellular telephone also has a screen within the body and connected to the central processing unit for displaying a second plurality of data wherein the second plurality of data is derived from the first plurality of data.

In another embodiment of the cellular telephone, the biometric port has a scanning device connected to the central processing unit which identifies the individual.

In another embodiment, the cellular telephone also has an antenna extending beyond the body and connected to the central processing unit for communicating with the cellular system.

In another embodiment, the cellular telephone also has an input unit within the body and connected to the central processing unit for entering at least one data from the first plurality of data relating to a lifestyle of the individual.

In another embodiment, the cellular telephone also has cursor extending beyond the body and connected to the central processing unit for navigating a screen menu.

In another embodiment, the cellular telephone also has a microphone within the body and connected to the central processing unit for receiving audio signals.

In another embodiment, the cellular telephone also has a speaker within the body and connected to the central processing unit for producing audio signals.

In another embodiment of the cellular telephone, the central processing unit stores mathematical algorithms for analyzing the first plurality of data.

In an embodiment of the present invention, a method for evaluating health and wellness of an individual using an electronic device having a central processing unit connected to a biometric port, an input unit, a memory unit, a tablet and a screen is provided. The method has the steps of: receiving a first plurality of data from the biometric port wherein the first plurality of data relates to physiological information of the individual; entering the first plurality of data wherein the first plurality of data relates to a lifestyle of the individual; analyzing the first plurality of data; storing the first plurality of data; and displaying a second plurality of data wherein the second plurality of data is derived from the first plurality of data.

In another embodiment, the method further has the step of communicating with a cellular system through the electronic device.

In another embodiment, the method further has the step of displaying the second plurality of data wherein the second plurality of data is derived from comparing the first plurality of data with a third plurality of data wherein the third plurality of data relates to a baseline biostatistic.

It is, therefore, an advantage of the present invention to provide a system, apparatus and method by which to portably evaluate health and wellness of an individual.

A further advantage of the present invention is to provide a system, apparatus and method to receive physiological information.

A further advantage of the present invention is to provide a system, apparatus and method to evaluate entered information relating to lifestyle, such as, for example, diet and exercise.

A further advantage of the present invention is to provide a system, apparatus and method to communicate with a cellular system and/or a computer network.

A further advantage of the present invention is to provide a system, apparatus and method for storing, analyzing and/or displaying received physiological information and/or entered information relating to lifestyle.

A further advantage of the present invention is to provide a system, apparatus and method for non-invasively receiving physiological information through epidermal contact.

A further advantage of the present invention is to provide a system, apparatus and method having a tablet for entering information.

A further advantage of the present invention is to provide a system, apparatus and method having an output component for communicating with a computer network.

A further advantage of the present invention is to provide a system, apparatus and method having a biometric port for identifying multiple users.

A further advantage of the present invention is to provide a system, apparatus and method having an antenna for communicating with a cellular system.

A further advantage of the present invention is to provide a system, apparatus and method for comparing received physiological information as well as entered information relating to lifestyle to information relating to a baseline biostatistic.

Additional features and advantages of the present invention are described in, and will be apparent from, the detailed description of the presently preferred embodiments and from the figures. Furthermore, changes and modifications to the identified features and advantages may be made without departing from the spirit and scope of the present invention.

### BRIEF DESCRIPTION OF THE FIGURES

FIG. 1 illustrates a diagram of an embodiment of a system that may evaluate an individual.

FIG. 2 illustrates a diagram of an embodiment of a cellular telephone that may evaluate an individual.

FIG. 3 illustrates a flowchart of an embodiment of a method that may evaluate an individual.

### DETAILED DESCRIPTION OF THE PRESENTLY PREFERRED EMBODIMENTS

The present invention provides a system, apparatus and method for evaluating the health and wellness of an individual. The system, apparatus and method is provided in an electronic device, such as, for example, a cellular telephone. The system, apparatus and method uses received and/or entered information relating to physiology, lifestyle, diet and/or exercise of an individual to evaluate his/her health and wellness. The system, apparatus and method may receive physiological information from an individual in a non-invasive manner and/or an invasive manner. The system, apparatus and method may store, analyze and/or display the received and/or entered information. The system, apparatus and method may also use the received and/or entered information in communication with a cellular system and/or a computer network.

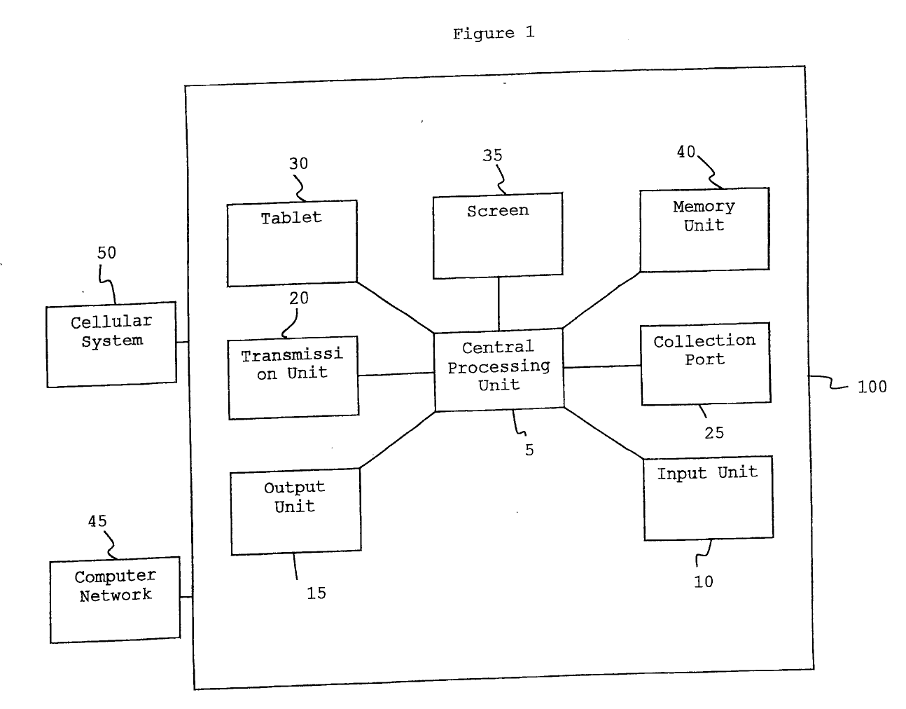

Referring to the figures, in which the reference numbers generally indicate identical, functionally similar or structurally similar elements, a system 100 for evaluating the health and wellness of an individual is generally illustrated in FIG. 1. The system 100 may generally have a central processing unit 5, a transmission unit 20, a collection port 25, a memory unit 40, a screen 35 and/or any similar elements that may be useful in evaluating the health and wellness of an individual.

The central processing unit 5 may perform myriad functions for the system 100. For example, the central processing unit 5 may function to guide the operation of, and may be connected to, the collection port 25, the screen 35, the transmission unit 20, the memory unit 40 and other elements of the system 100. In the system 100, the central processing unit 5 may also perform functions associated with, and may be connected to, an input unit 10, a tablet 30, an output component 15 as well as other elements of the system 100.

The collection port 25 of the system 100, which is connected to the central processing unit 5, may generally receive physiological information from an individual. This physiological information may be data that relates to a first plurality of data. The collection port 25 may receive this information through epidermal contact. The capability to receive this physiological information through epidermal contact may avoid the invasiveness commonly associated with receiving information of this type. For example, the collection port 25 may receive such information as pulse rate, blood pressure, skin temperature, blood sugar level, glucose level, body fat and/or similar physiological information. The collection port 25 may be comparable in functionality and structure to a biometric port. These biometric ports are commonly available in personal identification systems.

In an embodiment, the collection port 25 of the system 100 may receive physiological information from an individual through an invasive manner. The collection port 25 may collect a blood sample and other physiological information via, for example, a needle, laser, tube and/or other instrument (not shown) that may be apparent to those skilled in the art. The instrument may be stored in a recessed area within the collection port 25. The instrument may attach to a device (not shown) which may convert the blood sample and other physiological information into data to be analyzed by the central processing unit 5. The device may also contain a storage unit for the collection of wastes, such as, for example, blood or other bodily wastes, which may have been used to obtain the physiological information. The instrument may attach to the device via a spring, lever or the like, which may allow the instrument to penetrate the skin of the individual.

The individual may enter a command on the screen 35. The central processing unit 5 may then deliver a command to release the instrument. Alternatively, the collection port may contain sensors, which may automatically detect the finger or other body part of the individual. If the sensor is triggered, the device may then release the instrument. After the instrument is released, the blood sample and other physiological information collected may be converted to data via the device. The data may then be analyzed by the central processing unit 5. After the device analyzes the physiological information, the wastes may be disposed from the system 100.

The screen 35 of the system 100 may prompt an individual to provide his/her physiological information through the collection port 25. Physiological information that may be received through the collection port 25 may be analyzed via mathematical algorithms stored in the central processing unit 5 and/or the memory unit 40. These mathematical algorithms may standardize the physiological information into a commonly recognizable type, such as, for example, a blood pressure measurement in terms of millimeters of mercury. This standardized information may be data that relates to a second plurality of data. Such data may be reviewed by an individual through the screen 35. The memory unit 40 may also store this data, and its associated data from the first plurality of data, for review by the user.

The memory unit 40 of the system 100, which is connected to the central processing unit 5, may store information that is received, entered, displayed, analyzed and/or the like. The memory unit 40 may also store multiple pluralities of data, such as, for example, a first plurality, a second plurality and a third plurality. These pluralities of data may be received, entered, displayed, analyzed and/or the like. In addition, the pluralities of data may also be stored in the memory unit 40 prior to use of the system 100 by an individual. Such pluralities of data that may be stored prior to use include mathematical algorithms and baseline biostatistics. Baseline biostatistics are physiological standards, such as, for example, a body temperature of 37 degrees Celsius, from which received physiological information and the like may be compared.

The screen 35 of the system 100, which is connected to the central processing unit 5, may display a variety of information to the user. For example, the screen 35 may display information that is received, entered, stored, analyzed and/or the like. The screen 35 may also display numerous menus to the user. Such menus may be those that are commonly associated with a cellular telephone, such as, for example, settings and sounds. The menus displayed on the screen 35 may relate to prompts for receiving and/or entering information. Additionally, the screen 35 may also display multiple pluralities of data, such as, for example, a first plurality, a second plurality and a third plurality. These pluralities of data may be received, entered, stored, analyzed and/or the like.

The system 100 may also generally have an input unit 10 connected to the central processing unit 5 for entering information relating to lifestyle, such as, for example, diet and exercise. This information may be data that relates to a first plurality of data. For example, an individual may enter information relating to a type of snack. An individual may also enter information regarding the quantity of servings of a snack type which are consumed. Similarly, an individual may enter information on a type and/or duration of an activity that is performed. Such information offers a basis for feedback from the system 100 providing an individual with guidance on his/her decisions regarding diet and exercise.

The input unit 10 may be similar to a numeric, alpha-numeric, word recognition, character recognition and/or equivalent type keypad. Information may be entered through the input unit 10 according to queries displayed on the screen 35. The input information may be analyzed via mathematical algorithms stored in the central processing unit 5 and/or the memory unit 40. These mathematical algorithms may standardize the entered information into a commonly recognizable type, such as, for example, a type that is generally available in a food label having, to identify a few, caloric and fat quantities. The mathematical algorithms may also provide standardized exercise information similar to that displayed on various fitness equipment, such as, for example, a treadmill. This standardized information may be data that relates to a second plurality of data. Such data may be reviewed by an individual through the screen 35. The memory unit 40 may also store this data, and its associated data from the first plurality of data, for review by the user.

The system 100 may also generally have a tablet 30 connected to the central processing unit 5 for entering information including information relating to lifestyle, such as, for example diet and exercise. Information entered into the tablet 30 regarding lifestyle may generally be of the type associated with that entered into the input unit 10. This information may also be data that relates to a first plurality of data. Moreover, this lifestyle information may be analyzed via mathematical algorithms stored in the central processing unit 5 and/or the memory unit 40 in a manner consistent with the standardization associated with the input unit 10. The tablet 30 may be generally similar in function and structure to those character tablets used with personal digital assistants, tablet computers, handheld devices, mobile units and the like. In functionality, the tablet 30 has the character recognition capabilities associated with such devices and the capability to display entered information through the screen 35 of the system 100. For example, a character is entered into the tablet 30 through a finger and/or stylus. During use, an individual may decide to enter such information into the tablet 30 as personal notes, tasks or a grocery list. The central processing unit 5 may provide the character recognition capabilities associated with the tablet 30. The memory unit 40 may also store information that is entered into the tablet 30 for review by the user.

The transmission unit 20 of the system 100 may also generally connect to the central processing unit 5 for communicating with a cellular system 50 which may be external to the system 100. The cellular system 50 may be of a conventional type such as that associated with a cellular telephone network. The transmission unit 20 may wirelessly communicate with the cellular system 50. This communication offers a remote individual, such as, for example, a healthcare provider, the ability to review information that may be received, entered, displayed, stored, analyzed and/or the like in association with the system 100. Additionally, in communicating with the transmission unit 20, a healthcare provider may be able to provide information to a user of the system 100 regarding his/her health or wellness. Communication in this manner may provide a basis for integrated healthcare such that healthcare providers may regularly be able to monitor and evaluate the health and wellness of a patient based on information that may have previously been inaccessible. Such evaluations from a healthcare provider may be displayed on the screen 35 of the system 100.

The system 100 may also generally have an output component 15 connected to the central processing unit 5 for communicating with a computer network 45. The output component 15 may provide for communication with a computer network 45 in a manner similar to the communication of the system 100 with a cellular system 50. Specifically, the computer network 45 is external to the system 100 and there is communication of received, entered, displayed, stored, analyzed and/or the like information from the system 100 to the computer network 45. The computer network 45 may also provide health and wellness evaluations, based on communicated information, that may be useful to the user of the system 100. Such evaluations from the computer network 45 may be displayed on the screen 35 of the system 100.

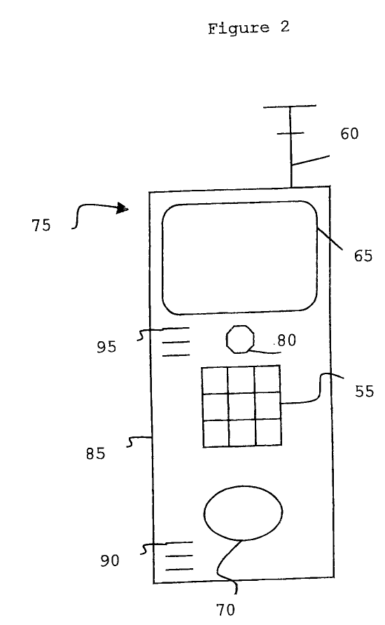

Referring to FIG. 2, a cellular telephone 75 for evaluating health and wellness of an individual is generally illustrated. The cellular telephone 75 may have the functional and structural elements of the system 100, generally illustrated in FIG. 1, within an exterior 85 of the cellular telephone 75. The exterior 85 of the cellular telephone 75 may be plastic, titanium or any other composition. Additionally, the exterior 85 may define a body of the cellular telephone 75 which may be of any form or type, such as, for example, a cellular telephone with an axis that pivots.

Within the body of the cellular telephone 75 may be a central processing unit, a biometric port 70, a memory unit, a screen 65 and/or any similar elements that may be useful in evaluating the health and wellness of an individual. The biometric port 70, memory unit and/or screen 65 of the cellular telephone 75 may be connected to the central processing unit. Moreover, the central processing unit, the memory unit and/or the screen 65 may be similar in functionality and structure to the corresponding elements generally described in system 100, illustrated in FIG. 1. The cellular telephone 75 may also have an input unit 55 within the body of the cellular telephone 75 and connected to the central processing unit. The input unit 55 is functionally and structurally similar to that generally described in the system 100.

The biometric port 70 of the cellular telephone 85 may generally receive physiological information from an individual. This physiological information may be data that relates to a first plurality of data. The biometric port 70 may receive this information through epidermal contact. The capability to receive this physiological information through epidermal contact may avoid the invasiveness commonly associated with receiving information of this type. For example, the biometric port 70 may receive such information as pulse rate, blood pressure, skin temperature, blood sugar level, glucose level, body fat and/or similar physiological information. The biometric port 70 may also have a scanning device connected to the central processing unit for identifying users of the cellular telephone 85. The scanning device may be similar in function and structure to those devices commonly available in personal identification systems.

In an embodiment, the cellular telephone 75 may enable the individual to track intake of food consumed. A name and/or type of the food consumed by the individual may be entered into the cellular telephone 75 for evaluation. In an example, the individual may monitor an amount of calories consumed during a given period. In another example, the individual may monitor an amount of protein consumed. The individual may also monitor, for example, an amount of carbohydrates or fat consumed.

In an embodiment, the cellular telephone 75 may be programmed with a reminder feature. The individual may require, for example, a reminder to take a medication. To that end, the individual may enter a time into the cellular telephone 75, for example, at which the individual is required to take the medication. The individual may also enter a message which may be displayed at the time the medication is to be taken, such as, for example, “Take your medication.” The reminder feature may provide the individual with an alert and/or may display the message at the time the medication is to be received.

The cellular telephone 85 may also have an antenna 60 that extends beyond the body of the cellular telephone 85. The antenna 60 may be similar in functionality and structure to the transmission unit 20 generally described in system 100, illustrated in FIG. 1. In addition, the antenna 60 may also be connected to the central processing unit of the cellular telephone 85 for communicating with a cellular system. The antenna 60 may also be movable such that the antenna 60 may be able to elongate and/or retract. Communication through the antenna 60 offers a remote individual, such as, for example, a healthcare provider, the ability to review information that may be received, entered, displayed, stored, analyzed and/or the like in association with the cellular telephone 85. Additionally, in communicating with the cellular telephone 85, a healthcare provider may be able to provide information to a user of the cellular telephone 85 regarding his/her health or wellness. Communication in this manner offers a healthcare provider the ability to monitor and evaluate the health and wellness of a patient based on information that may have previously been inaccessible. Such evaluations from a healthcare provider may be displayed on the screen 65 of the cellular telephone 85.

The cellular telephone 85 may also have a cursor 80 that extends beyond the body of the cellular telephone 85. The cursor 80 may be also be connected to the central processing unit of the cellular telephone 85 and be capable of navigating through any menu displayed on the screen 65. Such menus may be those that are commonly associated with a cellular telephone, such as, for example, settings and sounds. The cursor 80 may function in a manner similar to that of a computer cursor. This functionality may offer a user the ability to efficiently navigate a menu displayed on the screen 65 of the cellular telephone 85.

The cellular telephone 85 may also have a microphone 90 within the body of the cellular telephone 85. The microphone 90 may also be connected to the central processing unit of the cellular telephone 85 and be capable of receiving audio signals. Such signals may be those that are commonly associated with a cellular telephone, such as, for example, voice signals. The microphone 90 may offer a user the ability to perform voice transmissions in association with the antenna 60 of the cellular telephone 85.

The cellular telephone 85 may also have a speaker 95 within the body of the cellular telephone 85. The speaker 95 may also be connected to the central processing unit of the cellular telephone 85 and be capable of producing audio signals. Such signals may be those that are commonly associated with a cellular telephone, such as, for example, voice signals. The speaker 95 may offer a user the ability to receive voice transmissions in association with the antenna 60 of the cellular telephone 85.

In an embodiment, the cellular telephone 75 may be programmed to have a calculator mode. The individual may enter values and/or commands into cellular telephone 75 during the calculator mode via the input unit 10. The cellular telephone 75 may enable the individual to calculate, for example, an amount spent for grocery items. In an example, the individual may obtain a price for a first grocery item while shopping and may enter the price into the cellular telephone 75. The individual may then obtain a price for a second grocery item and enter the price into the cellular telephone 75 to receive a subtotal for the groceries purchased. The calculations performed during operation of the calculator mode should not be understood as limited to grocery-related calculations, but may include any type of calculating functions.

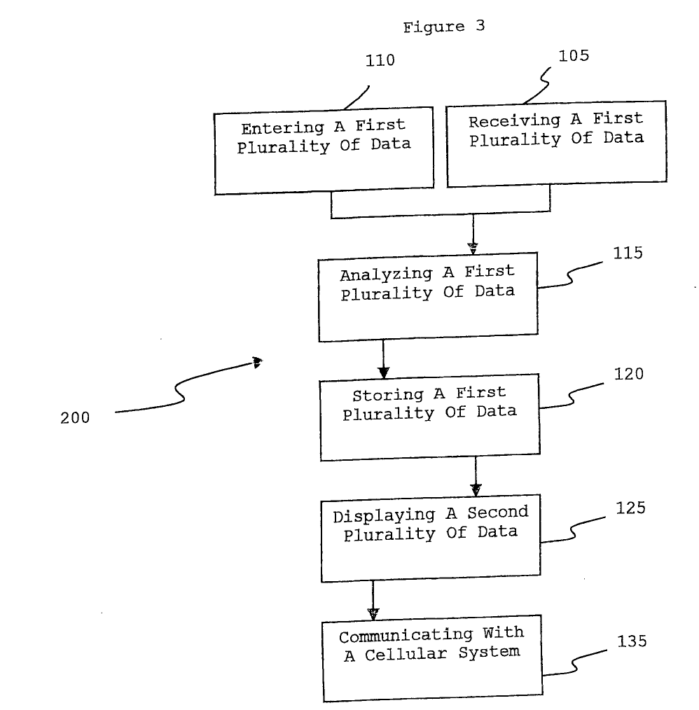

Referring to FIG. 3, a diagram of a method 200 for evaluating the health and wellness of an individual is generally illustrated. The method 200 may generally have the functional and structural elements of the system 100 and the cellular telephone 85, illustrated in FIG. 1 and FIG. 2, respectively. For example, the method 200 may have an electronic device with a central processing unit connected to a biometric port, an input unit, a memory unit, a tablet and/or a screen as generally described for the system 100 and/or the cellular telephone 85, as well as any similar elements that may be useful in evaluating the health and wellness of an individual. Furthermore, the method 200 may have steps that include: receiving 105 physiological information through the biometric port; entering 110 lifestyle information through the input unit; analyzing 115 the physiological and/or lifestyle information; storing 120 the physiological and/or lifestyle information and displaying 125 the physiological and/or lifestyle information that may be analyzed. As illustrated, receiving 105 physiological information and entering 110 lifestyle information may be connected to and precede the step of analyzing 115 the physiological and/or lifestyle information. Correspondingly, analyzing 115 the physiological and/or lifestyle information may be connected, precede and be subsequent to the step of storing 120 the physiological and/or lifestyle information and displaying 125 the physiological and/or lifestyle information.

In receiving 105 physiological information, the information may be received through the biometric port and may be data that relates to a first plurality of data. Similarly, in the step of entering 110 lifestyle information, the information may be entered through the input unit and may also be data that relates to the first plurality of data. Analyzing 115 the physiological and/or lifestyle information and storing 120 such may correspond to an analysis and storage, respectively, of the first plurality of data. The analysis of the first plurality of data, referred to as the step of analyzing 115, may establish a second plurality of data. This second plurality of data may, for example, be a standardization of the physiological and/or lifestyle information such that the information is displayed as a commonly recognizable type like that generally described for the system 100 and/or the cellular telephone 85. The step of displaying 125 physiological and/or lifestyle information may be through the screen of the electronic device. The displaying 125 step may also provide to the user information from the steps of receiving 105, entering 110, storing 120, analyzing 115 and/or the like.

The displaying 125 step of the method 200 may also involve comparing the first plurality of data with a third plurality of data. The third plurality of data is used for comparison with the first plurality of data and may include baseline biostatistics. Baseline biostatistics are physiological standards, such as, for example, a body temperature of 37 degrees Celsius. These baseline biostatistics may be available through the method 200 step of storing 120.

In the entering 110 step of the method 200, information relating to lifestyle may also be entered through a tablet of the electronic device. As generally described for the system 100 and the cellular telephone 85, the tablet may also be used for entering 110 such information as personal notes, tasks and/or a grocery list. This information may also be data that relates to a first plurality of data. The tablet may be generally similar in function and structure to those character tablets used with personal digital assistants, tablet computers, handheld devices, mobile units and the like.

Communicating 135 with a cellular system through the electronic device may also be a step in the method 200. This step of communicating 135 may be connected, precede and be subsequent to the analyzing 115 step of the method 200. The communicating 135 step may also be similar to that generally described for the system 100 and cellular telephone 85. Specifically, communicating 135 with a cellular system offers a remote individual, such as, for example, a healthcare provider, the ability to review information within the steps of receiving 105, entering 110, displaying 125, storing 120, analyzing 115 or the like. Additionally, in the step of communicating 135, a healthcare provider may be able to provide information to a user of the electronic device regarding his/her health or wellness. This information may be provided to the user through the method 200 step of displaying 125.

In another embodiment, the system 100 may be used as part of a program to provide health-related services to the individual. For example, the individual may be enrolled in a weight-reduction program. During the course of the program, the individual may receive messages which are sent to the cellular telephone 75. The messages may be sent, for example, in the form of text messages, electronic mail, facsimilie, or other modes of communication. Content of the messages may include instructions for following the program or, for example, messages of encouragement to continue performance of the program.

It should be understood that although various embodiments of the present invention have been described, the embodiments are presented by way of example and not limitation. It should further be understood that various modifications or changes to the presently preferred embodiments described herein will be apparent to those skilled in the art. Thus, the breadth and scope of the present invention should not be limited by either any of the described exemplary embodiments or by changes or modifications that may be made without departing from the spirit and scope of the present invention. The breadth and scope of the present invention should be defined only in accordance with the following claims and equivalents thereof.

1. A system for evaluating health and wellness of an individual, the system comprising:
a central processing unit for analyzing a first plurality of data;
a transmission unit connected to the central processing unit for communicating with a cellular system;
a collection port connected to the central processing unit for receiving physiological information from the individual;
a memory unit connected to the central processing unit for storing the first plurality of data wherein the first plurality of data relates to the physiological information; and
a screen connected to the central processing unit for displaying a second plurality of data wherein the second plurality of data is derived from the first plurality of data.

2. The system of claim 1 further comprising:
an input unit connected to the central processing unit for entering the first plurality of data.

3. The system of claim 1 wherein the first plurality of data relates to a diet of the individual.

4. The system of claim 1 wherein the first plurality of data relates to an activity performed by the individual.

5. The system of claim 1 wherein the collection port non-invasively receives the physiological information through epidermal contact of the individual.

6. The system of claim 1 wherein the physiological information is selected from the group consisting of pulse rate, heart rate, blood pressure, glucose level, blood sugar level, skin temperature, body fat and combinations thereof.

7. The system of claim 1 wherein the collection port
invasively receives the physiological information through epidermal contact of the individual.

8. The system of claim 1 wherein the central processing unit stores mathematical algorithms for analyzing the first plurality of data.

9. The system of claim 1 further comprising:
an output component connected to the central processing unit for communicating with a computer network.

10. A cellular telephone having an exterior defined by a body that receives and transmits physiological information related to evaluating health and wellness of an individual, the cellular telephone comprising:
a central processing unit within the body for analyzing a first plurality of data;
a biometric port within the body and connected to the central processing unit for receiving the physiological information from the individual;
a memory unit within the body and connected to the central processing unit for storing the first plurality of data wherein the first plurality of data relates to the physiological information; and
a screen within the body and connected to the central processing unit for displaying a second plurality of data wherein the second plurality of data is derived from the first plurality of data.

11. The cellular telephone of claim 10 wherein the biometric port has a scanning device connected to the central processing unit which identifies the individual.

12. The cellular telephone of claim 10 further comprising:
an antenna extending beyond the body and connected to the central processing unit for communicating with the cellular system.

13. The cellular telephone of claim 10 further comprising:
an input unit within the body and connected to the central processing unit for entering the first plurality of data relating to a lifestyle of the individual.

14. The cellular telephone of claim 10 further comprising:
a cursor extending beyond the body and connected to the central processing unit for navigating a screen menu.

15. The cellular telephone of claim 10 further comprising:
a microphone within the body and connected to the central processing unit for receiving audio signals.

16. The cellular telephone of claim 10 further comprising:
a speaker within the body and connected to the central processing unit for producing audio signals.

17. The cellular telephone of claim 10 wherein the central processing unit stores mathematical algorithms for analyzing the first plurality of data.

18. A method for evaluating health and wellness of an individual using an electronic device having a central processing unit connected to a biometric port, an input unit, a memory unit, a tablet and a screen, the method comprising the steps of:
receiving a first plurality of data from the biometric port wherein the first plurality of data relates to physiological information of the individual;
entering the first plurality of data wherein the first plurality of data relates to a lifestyle of the individual;
analyzing the first plurality of data;
storing the first plurality of data; and
displaying a second plurality of data wherein the second plurality of data is derived from the first plurality of data.

19. The method of claim 18 further comprising the step of:
communicating with a cellular system through the electronic device.

20. The method of claim 18 further comprising the step of:
displaying the second plurality of data wherein the second plurality of data is derived from comparing the first plurality of data with a third plurality of data wherein the third plurality of data relates to a baseline biostatistic.
